(19) United States
(12) Patent Application Publication
(10) Pub. No.: US 2008/0125746 A1
(43) Pub. Date: May 29, 2008

(54) Collection catheter and method

(76) Inventors: James Edward Shapland, Vadnais Heights, MN (US); Tuan Minh Doan, Burnsville, MN (US)

Correspondence Address: MERCHANT & GOULD PC, P.O. BOX 2903, MINNEAPOLIS, MN, 55402-0903, US

(21) Appl. No.: 11/506,257

(22) Filed: Aug. 18, 2006

(51) Int. Cl. A61M 31/00; A61M 5/168

(52) U.S. Cl. 604/508; 604/247

(57) ABSTRACT

A catheter for collecting fluid (such as blood) from a vessel (such as a coronary sinus) of a patient has a distal end with a fluid inlet to receive a blood and at least one fluid outlet disposed proximally to the fluid inlet. An occlusion member is positioned between the fluid inlet and the fluid outlet. The catheter is placed in a blood vessel with the fluid inlet disposed to receive antegrade flow. The blood vessel is occluded with the occlusion member. Flow through the catheter is passed through the fluid outlet and back into the vessel proximal to the occlusion member. A valve selectively opens and closes the fluid outlet.

### I. BACKGROUND OF THE INVENTION

1. Field of the Invention

This invention pertains to catheters for collecting fluid from a blood vessel of a patient. More particularly, this invention pertains to a catheter for selectively controlling the collection of blood or other fluid through the catheter.

2. Description of the Prior Art

Collection catheters are well known for collecting blood or other fluids from blood vessels of a patient. For example, International Publication No. WO 2005/082440 A1 describes collection catheters for collecting blood from a coronary sinus or other coronary vein in a perfusion system.

In addition to collecting blood in a perfusion process, blood may be collected from a coronary vein in an angiography procedure. Such procedures are used for assessing patency of coronary arteries. In such a procedure, a contrast medium is injected into a coronary artery proximal to a suspected site of a coronary obstruction. Such contrast media may have significant health risks if permitted to flow systemically to the patient's organs. For example, renal dysfunction or failure may occur from such systemic delivery of a contrast media. Such failure is referred to as “contrast-induced nephropathy” or CIN. Schräder, “Contrast Media-Induced Renal Failure: And Overview”, Journal of Interventional Cardiology, Vol. 18, No. 6, pages 417-423 (2005). A number of different techniques in catheter designs have been suggested for collecting contrast media. Examples of such are shown in U.S. Pat. No. 6,554,819 to Reich issued Apr. 29, 2003; U.S. Patent Application Publication No. US 2002/0099254 A1 to Movahed published Jul. 25, 2002; U.S. Patent Application Publication No. US 2005/0256441 A1 to Lotan et al., published Nov. 17, 2005 and U.S. Patent Application Publication No. US 2006/0013772 A1 to LeWinter et al., published Jan. 19, 2006. A contrast removal system is also described in Michishita, et al. “A Novel Contrast Removal System From The Coronary Sinus Using An Absorbing Column During Coronary Angiography In A Porcine Model”, Journal of the American College of Cardiology, Vol. 47, No. 9 (2006).

It is an object of the present invention to provide a method and apparatus for collection of a fluid from a coronary vessel.

### II. SUMMARY OF THE INVENTION

According to a preferred embodiment of the present invention, a method and apparatus are disclosed for collecting fluid from a blood vessel (such as a coronary sinus) of a patient. The method includes use of a catheter having a distal end with a fluid inlet to receive flow from a blood vessel into a lumen of the catheter. The catheter further has at least one fluid outlet through the catheter and in communication with the catheter lumen. The fluid outlet is disposed proximally to the fluid inlet. An occlusion member is positioned between the fluid inlet and the fluid outlet. The method includes placing the catheter in the blood vessel with the fluid inlet disposed to receive antegrade flow from the blood vessel. The blood vessel is occluded with the occlusion member to substantially block flow between opposing surfaces of the vessel and the catheter. Flow through the lumen is passed through the fluid outlet and back into the vessel proximal to the occlusion member. In further embodiments of the invention, a valve is provided for selectively opening and closing the fluid outlet. Proximal and distal are used herein with reference to the catheter structure and not to the direction of normal blood flow in the blood vessel.

### III. BRIEF DESCRIPTION OF THE DRAWINGS

FIG. 1 is a perspective view of a distal end of a collection catheter according to the present invention;

FIG. 2 is a cross-sectional view of the catheter of FIG. 1 taken along line 2-2 of FIG. 1;

FIG. 3 is a cross-sectional view of the catheter of FIG. 1 with a balloon shown in an expanded state and taken along line 3-3 of FIG. 1;

FIG. 4 is a schematic side sectional view of the catheter of FIG. 1 in a coronary sinus and showing a first embodiment of a valve in an open position;

FIG. 5 is the view of FIG. 4 showing the valve in a closed position;

FIG. 6 is the view of FIG. 2 showing a second embodiment of a valve shown rotated to an open position;

FIG. 7 is the view of FIG. 6 with the valve shown rotated to a closed position;

FIG. 8 is the view of FIG. 1 showing a still further alternative embodiment of a valve;

FIG. 9 is a view similar to that of FIG. 4 showing the catheter of FIG. 8 with a valve in an open position;

FIG. 10 is the view of FIG. 9 showing the valve in a closed position;

FIG. 11 is a schematic representation of use of the collection catheter of the present invention in a perfusion system for injecting perfusate or contrast media into a coronary artery and showing a valve of the collection catheter in a open position; and

FIG. 12 is the view of FIG. 11 with a valve shown in a closed position.

### IV. DESCRIPTION OF THE PREFERRED EMBODIMENT

Referring now to the several drawing figures in which identical elements are numbered identically throughout, a description of a preferred embodiment of the present invention will now be provided.

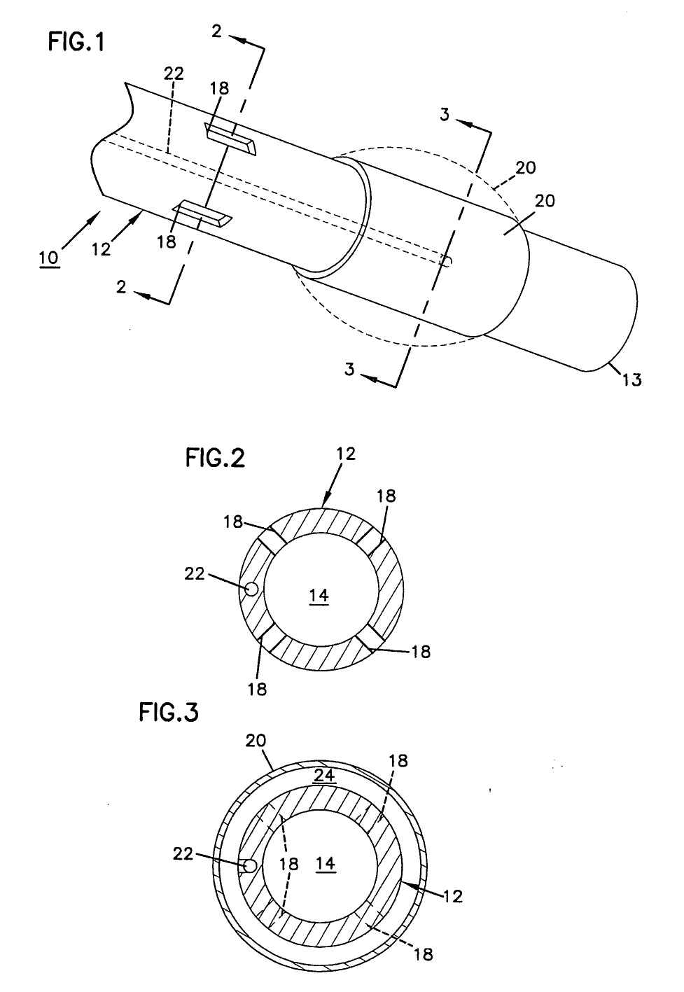

FIG. 1 shows a collection catheter 10. In a preferred embodiment, the collection catheter 10 is used to collect blood flow laden with a perfusate or a contrast media which had been injected into a coronary artery for angiography or similar procedures. The collection catheter 10 is to collect blood flow from the coronary sinus before such blood flow can pass systemically to the remainder of the patient's body. While such is a preferred embodiment, the apparatus of the present invention can be used in any blood collection technique including that described in the aforementioned International Publication No. WO 2005/082440 A1 (incorporated herein by reference) as well as any dialysis or similar blood collection system. Accordingly, the reference to coronary sinus is illustrative only and can include any other blood vessel.

With initial reference to FIGS. 1-5, the catheter 10 is shown. The catheter 10 includes a catheter body 12 terminating at a distal end 13. The catheter body 12 is formed from any suitable material for catheters as is well known in the art with the catheter being a long flexible tube adapted for insertion and advancement through the vasculature of a patient.

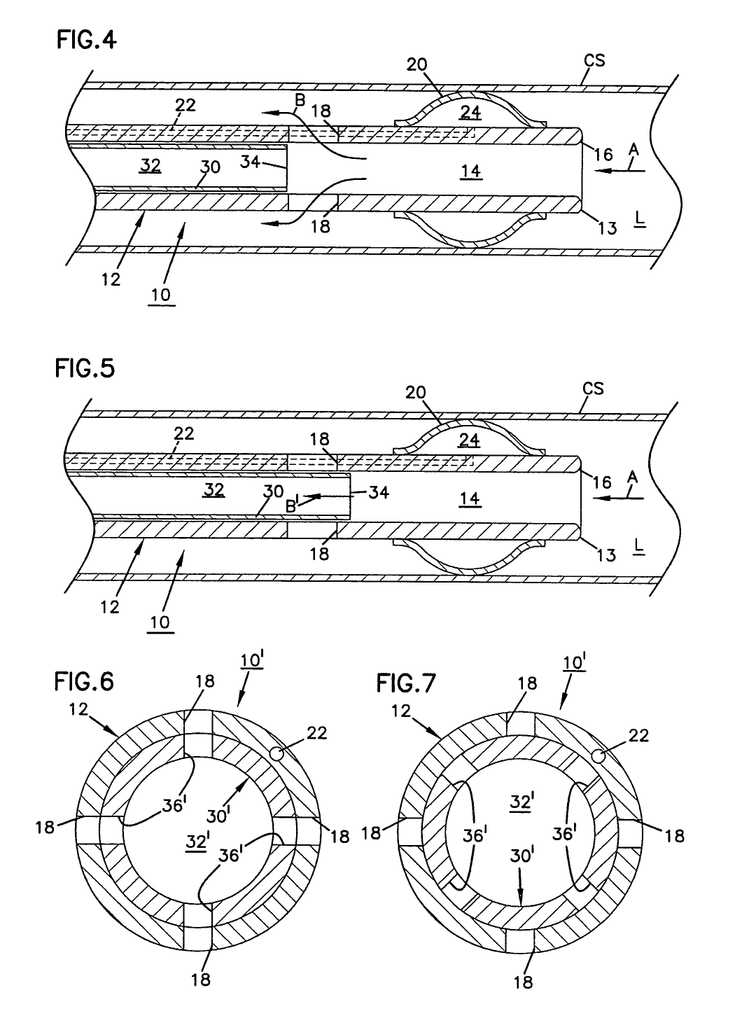

In FIGS. 4 and 5, the catheter is shown residing within the lumen L of a coronary sinus CS. As will be more thoroughly described, such use is for a preferred embodiment with a catheter 10 is used to collect perfusate from a treatment as shown in International Publication No. WO 2005/082440 A1. The catheter 10 can also be used to collect a contrast media in an angiography or similar procedure. However, as will be apparent to one of ordinary skill in the art, the catheter 10 can be used in other blood vessels for collection of fluids from the vessel in a wide variety of procedures.

Shown best in FIGS. 4 and 5, the catheter body 12 is hollow having a central lumen 14. The distal end 13 has an opening 16 to act as an inlet opening for admitting the blood flow into the lumen 14. In the Figures the normal direction of net blood flow (referred to as “antegrade” flow) is shown by arrow A.

The catheter body 12 has fluid outlets 18 in the form of holes 18 which extend through the wall thickness of the catheter body 12 proximal to the distal end 13. As used throughout this application, the terms “proximal” and “distal” refer to relative positioning on the catheter 10, the end 13 being the most distal end, and proximal being a location along the catheter away from the distal end 13.

An occlusion member is provided in the form of a balloon 20. The balloon 20 is positioned between the inlet opening 13 and the outlet openings 18. It will be appreciated that inflatable balloons for catheters are well known in the art. An inflation lumen 22 is provided within the wall thickness of the catheter body 12. The inflation lumen 22 communicates with the interior 24 of the balloon 20. Accordingly, an inflation fluid (such as a gas or liquid) can be admitted into or withdrawn from the interior 24 to inflate or deflate the balloon 20 as desired.

The catheter 10 includes a valve for selectively opening and closing the fluid outlets 18. In the embodiment of FIGS. 4 and 5, the valve 30 is a second catheter sized to be slidably received within the lumen 14 of the catheter body 12.

The valve catheter 30 has an internal lumen 32 extending along its length and exposed through an open distal end 34. The catheter valve 30 is moveable between an open position and a closed position.

In the open position (shown in FIG. 4), the catheter 30 is retracted relative to catheter body 12. So retracted, the distal end 34 of the catheter valve 30 is positioned proximal to the locations of the fluid outlets 18. The catheter 30 has an outside diameter approximately equal to the inside diameter of the lumen 14 to minimize fluid leakage between opposing surfaces of the catheter 30 and catheter body 12.

FIG. 5 shows the catheter valve 30 in a closed position. The catheter valve 30 is moved distally toward end 13 such that end 34 is advanced beyond the outlets 18 with the catheter 30 covering and obstructing the outlets 18.

In the open position of FIG. 4, fluid entering the lumen 14 through inlet 13 may freely pass through outlet 18 as indicated by arrows B. In the closed position of FIG. 5, the fluid within the lumen 14 is blocked from passing through outlets 18. Instead, flow (arrow B′) is through lumen 32 and out of a proximal end of the catheter 30 (not shown).

FIGS. 4 ad 5 show an embodiment where the valve is a sliding catheter 30. FIGS. 6 and 7 illustrate an alternative embodiment where the valve is a rotating catheter 30′. In the collection catheter 10′ of FIGS. 6 and 7, the valve body 12 is the same as in the embodiments of FIGS. 4 and 5.

In the embodiments of FIGS. 6 and 7, the catheter valve 30′ rotates about its longitudinal axis between an open position shown in FIG. 6 and a closed position shown in FIG. 7.

The catheter valve 30′ has a plurality of openings 36′ formed through its wall. The number, size and the disposition of each of the openings 36′ are selected to correspond with the number, size and positioning of the outlets 18.

In the open position of FIG. 6, the openings 36′ are aligned with the outlets 18 for fluid within the lumen 32′ to flow through the outlets 36′ and through outlets 18. In the closed position of FIG. 7, the catheter valve 30′ is rotated 45 degrees (in the specific embodiment shown) relative to the catheter body 12. So rotated, the alignment of outlets 18 and openings 36′ are offset so that fluid is blocked from flowing from the lumen 32′ out of the outlets 18.

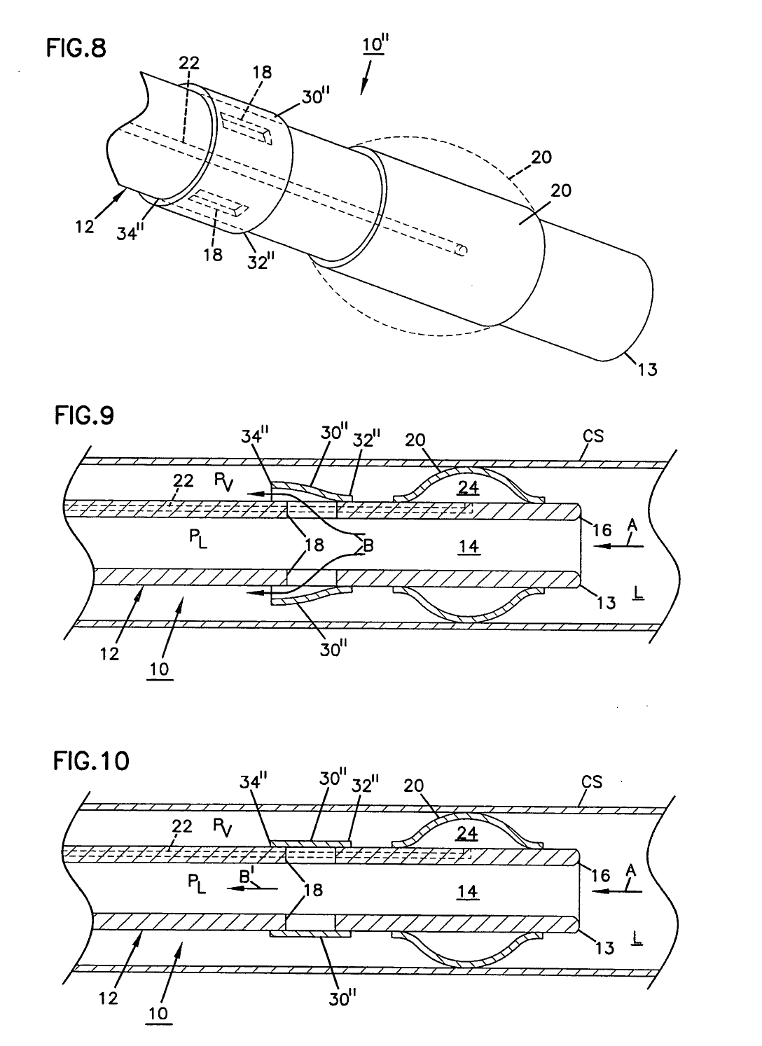

FIGS. 8-10 illustrate a still further embodiment of a collection catheter 10″. As in the previous embodiments, the catheter body 12 and its components are identical to the previously described embodiments and its elements are numbered identically through.

A valve 30″ is shown in the form of a sleeve surrounding the exterior of the catheter body 12 and covering the openings 18. A distal end 32″ of the valve 30′ is secured to the valve body 12. A proximal end 34″ is unattached to the catheter body 12. Sleeve 30″ is preferably a flexible resilient material such as silicone or the like.

As shown in FIG. 9, when the pressure P_{V} in the blood vessel CS is less than the pressure P_{L} in the lumen 14, the flow urges the valve 30″ open. The blood flows through the outlets 18 (arrow B). In the event of a reduction in pressure within the lumen 14 (such as coupling the lumen 14 to a source of a vacuum), all fluid flow (arrow B′) is drawn through the lumen 14 collapsing the sleeve 30 to cover openings 18.

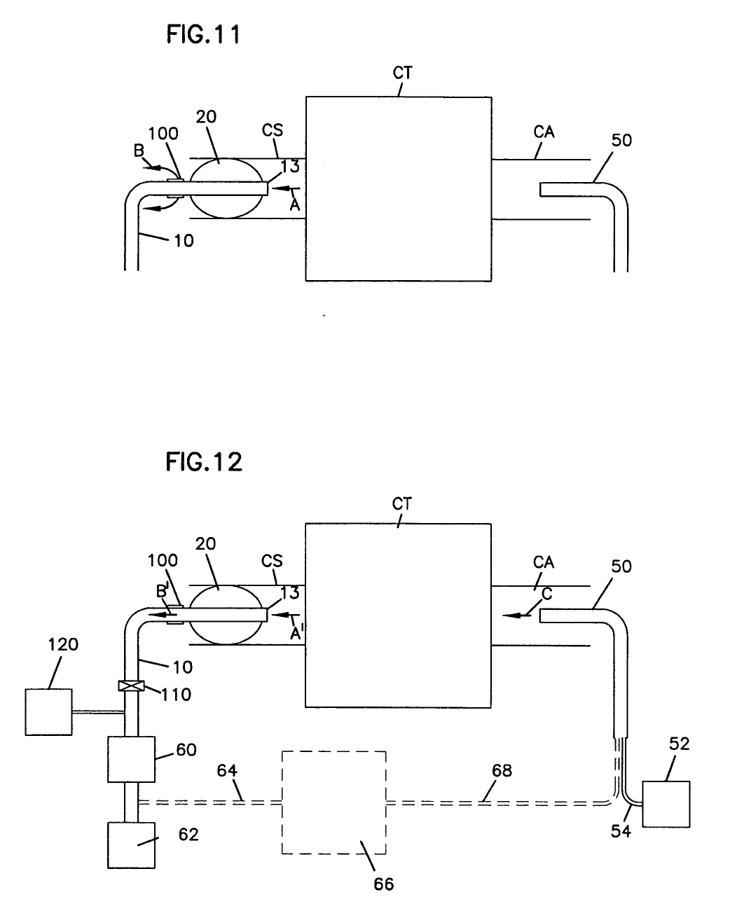

FIGS. 11 and 12 illustrate a use of the collection catheter 10. In FIGS. 11 and 12, cardiac tissue CT is schematically shown with a coronary artery CA as a source of oxygenated blood. A coronary sinus is shown for withdrawing blood from the cardiac tissue CT.

A delivery catheter 50 is shown in the coronary artery CA for delivery of any fluid into the coronary artery CA. The fluid may be a perfusate such as that described in the aforementioned International Publication No. WO 2005/082440 A1. The fluid may also be a contrast media for imaging procedures such as an angiography procedure. Shown only in FIG. 11, the injected fluid is shown in a source 52 with a tube 54 connecting the injected media to the delivery catheter 50. The media may be pumped or injected through syringe or otherwise delivered to the delivery catheter 50.

The collection catheter 10 of the present invention is shown in the coronary sinus with the balloon 20 inflated. The catheter 10 is shown connected to a pump or suction source 60.

The outlet of the suction source 60 is delivered to a reservoir 62 for collection of fluid for ultimate disposal. Alternatively, the fluid may be passed through a conduit (shown in phantom lines at 64) for delivery to any suitable treatment apparatus 66 for removal of undesired constituents within the collective fluid. For example, contrast media or perfusate can be removed in whole or in part from blood drawn through catheter 10. The treated blood is passed through a conduit 68 into the delivery catheter 50.

As shown in FIG. 11, the present apparatus is provided with a valve (generically numbered 100 to represent all embodiments described in this application). The valve 100 is in the open position when fluid from the source 52 is injected into the coronary artery CA (arrow C). When fluid is not so injected, the valve 100 is in the closed position (FIG. 12).

With the present invention, blood flow in the coronary sinus can be collected or permitted to pass through the coronary sinus by operation of the valve. The valve 100 may be opened or closed rapidly relative to other options such as inflating or deflating a balloon 20. The valve 100 may be in a closed position continuously during injection of a perfusate or contrast media. Alternatively, the valve 100 may be incrementally opened and closed to permit some of the perfusate or contrast media to pass through the coronary sinus. It is anticipated the valve is closed (FIG. 12) for about two to seven heart beats following contrast injection into the coronary artery to collect the contrast-laden blood flow. Then, it is opened (FIG. 11) to permit contrast-free blood flow from the heart.

In FIG. 12, a stop-cock 110 or other shut-off mechanism is provided in catheter 10 proximal to valve 100 and external to the patient's body for access by a physician or other operator. When flow is desired out of the valve 100 (FIG. 11), the stop-cock 110 is closed. When the valve 100 is closed (FIG. 12), the stop-cock 110 is open.

FIG. 12 also illustrates a pressure monitor 120 for monitoring pressure in the lumen of catheter 10. If too much suction is applied to the catheter 10, the coronary sinus CS can collapse. Monitoring pressure permits the physician to avoid such collapse.

Having described the present invention of the preferred embodiment, modifications and equivalents will become apparent to one of ordinary skill in the art. It is intended that such modifications and equivalents be included within the scope of the claims which are appended hereto.

We claim:

1. A method of collecting fluid from a blood vessel of a patient using a catheter having:
a. a distal end with a fluid inlet to receive flow of fluid from a blood vessel into a lumen of the catheter;
b. at least one fluid outlet through the catheter and in communication with the catheter lumen, the fluid outlet disposed proximally to the fluid inlet;
c. an occlusion member positioned between the fluid inlet and the fluid outlet;
the method comprising:
placing the catheter in the blood vessel with the fluid inlet of the catheter disposed to receive antegrade flow from the vessel;
occluding the vessel with the occlusion member to substantially block flow between opposing surfaces of the vessel and the catheter;
passing flow from the lumen through the fluid outlet and into the vessel proximal to the occlusion member.

2. A method according to claim 1 further comprising selectively opening and closing the fluid outlet.

3. A method according to claim 2 wherein the catheter includes a valve for opening and closing the fluid outlet, the method including selectively operating the valve.

4. A method according to claim 2 wherein the catheter includes a valve responsive to a differential in fluid pressure between the lumen and the vessel with the fluid outlet biased to one of an open and closed position and shifting to the other of the positions in response to a predetermined pressure differential, the method including altering a pressure in the lumen to open and close the valve.

5. A method according to claim 1 wherein the vessel is a coronary vein and the fluid is blood laden with a contrast medium.

6. An apparatus for collecting fluid from a blood vessel of a patient, the apparatus comprising:
an elongated flexible catheter body having an internal lumen and a distal end;
at least one fluid inlet to receive flow of fluid from a blood vessel into a lumen of the catheter;
said catheter adapted for the distal end to be advanced through the vessel with the an open distal end of the catheter disposed to receive antegrade flow from the vessel;
at least one fluid outlet through the catheter and in communication with the catheter lumen, the fluid outlet disposed proximally to the fluid inlet;
an occlusion member positioned between the fluid inlet and the fluid outlet and adapted to occlude the vessel to substantially block flow between opposing surfaces of the vessel and the catheter.

7. An apparatus according to claim 6 further comprising a valve for selectively opening and closing the fluid outlet.

8. An apparatus according to claim 7 wherein the catheter includes:
a valve body having a distal end movable between open and closed positions for opening and closing the fluid outlet;
the valve body having a proximal end coupled to the distal end for shifting the distal end the open and closed position in response to movement of the valve body proximal end.

9. An apparatus according to claim 7 wherein the valve is responsive to a differential in fluid pressure between the lumen and the vessel with the fluid outlet biased to one of an open and closed position and shifting to the other of the positions in response to a predetermined pressure differential.
